(19) United States
(12) Patent Application Publication
(10) Pub. No.: US 2026/0015430 A1
(43) Pub. Date: Jan. 15, 2026

(54) ANTIGEN BINDING PROTEIN SPECIFIC FOR TRANSMEMBRANE ACTIVATOR AND CAML INTERACTOR (TACI)

(71) Applicant: Agency for Science, Technology and Research, Singapore (SG)

(72) Inventors: Kong-Peng Lam, Singapore (SG); Shengli Xu, Singapore (SG); Jianxin Huo, Singapore (SG)

(21) Appl. No.: 19/127,906

(22) PCT Filed: Oct. 31, 2023
(86) PCT No.: PCT/SG2023/050725

§ 371 Date: May 7, 2025

(30) Foreign Application Priority Data
Nov. 8, 2022 (SG) 10202260012V

(51) Int. Cl. C07K 16/28; A61P 35/00

(52) U.S. Cl. CPC C07K 16/2878; A61P 35/00; C07K 16/2809; C07K 2317/24; C07K 2317/31; C07K 2317/33

(57) ABSTRACT

Provided herein is an antigen-binding protein that specifically binds and/or detects the transmembrane activator and calcium-modulator and cyclophilin ligand (CAML) interactor (TACI)/TNFRSF13B, wherein the antigen binding protein comprises a CDR sequence having GFSITSDYA (SEQ ID NO: 1) for CDRH1, ISYSGST (SEQ ID NO: 2) for CDRH2, ARVVSTSFDS (SEQ ID NO: 3) for CDRH3, ESVDNYGISF (SEQ ID NO: 4) for CDRL1, VAS (SEQ ID NO: 5) for CDRL2, and QQSKEVPYT (SEQ ID NO: 6) for CDRL3, or having amino acid sequences that are at least 85% identical to the sequences, or sequences having 1 or 2 amino acids different from the sequences thereof. Also provided herein are polynucleotides, vectors, and host cells thereof. Further provided herein arc pharmaceutical compositions and methods of treating a discasc, such as TACI-positive multiple mycloma.

### TECHNICAL FIELD

The present disclosure relates broadly to antigen binding protein that binds transmembrane activator and CAML interactor (TACI). The present disclosure also relates to therapeutic uses, compositions, and methods for producing said antigen-binding protein.

### BACKGROUND

The transmembrane activator and calcium-modulator and cyclophilin ligand (CAML) interactor (TACI) is a member of the tumor necrosis factor receptor (TNFR) superfamily and is also designated as TNFRSF13B. It was first identified as a binding partner of CAML protein in Jurkat T cells. TACI functions as a co-inducer of the nuclear factor of activated T cells (NF-AT) transcription factor. Cross-linking of TACI by anti-TACI antibodies also led to the activation of transcription factors AP-1 and NF-κB. Later on, together with B cell maturation antigen (BCMA), TACI was identified as a receptor that can bind B-cell activating factor (BAFF) and a proliferation-inducing ligand (APRIL), the two important B cell cytokines, and has been shown to play important roles in B cell activation and differentiation.

Although BAFF and APRIL are known to play important stimulatory roles in B cells, the initial characterization of TACI-deficient mice showed that TACI played an unexpected inhibitory role in B cell activation in vivo, as TACI−/− mice manifested splenomegaly with increased mature B cells. Subsequent studies demonstrated that TACI was important for BAFF- and APRIL-induced immunoglobulin (Ig) isotype switching to IgG1, IgA, and IgE in murine B cells. In addition, TACI was shown to be essential for the differentiation and survival of plasmablasts and plasma cells. Interestingly, TACI is expressed on multiple myeloma (MM) cells.

A recent study showed a chimeric antigen receptor (CAR) design using the receptor-binding domain of APRIL as the tumor antigen-targeting domain (APRIL-based chimeric antigen receptors; ACAR), which recognizes both BCMA and TACI on MM cells, could potentially achieve an improved clinical outcome for MM treatment. These results suggest that TACI is a potential target for developing immunotherapies against MM.

Therefore, there is a need to provide an alternative antigen-binding protein that has specificity for TACI.

### SUMMARY

In one aspect, there is provided an antigen-binding protein that specifically binds and/or detects the transmembrane activator and calcium-modulator and cyclophilin ligand (CAML) interactor (TACI)/TNFRSF13B, wherein the antigen binding protein comprises a CDR sequence having GFSITSDYA (SEQ ID NO: 1) for CDRH1, ISYSGST (SEQ ID NO: 2) for CDRH2, ARVVSTSFDS (SEQ ID NO: 3) for CDRH3, ESVDNYGISF (SEQ ID NO: 4) for CDRL1, VAS (SEQ ID NO: 5) for CDRL2, and QQSKEVPYT (SEQ ID NO: 6) for CDRH1, or having amino acid sequences that are at least 85% identical to the sequences, or sequences having 1 or 2 amino acids different from the sequences thereof.

In some examples, the antigen binding protein activates transcription factors AP-1 and/or NF-κB.

In some examples, the antigen binding protein is capable of modulating TACI-induced B cell activation and/or differentiation.

In some examples, the antigen binding protein is a multispecific antigen binding protein that binds and/or detects TACI and/or engages/activates other immune cells, optionally wherein the antigen binding protein is a bispecific antigen binding protein that binds and/or detects TACI and CD3.

In some examples, the antigen binding protein is a monoclonal antibody.

In some examples, the antigen binding protein is a neutralizing antibody and/or a humanized antibody.

In some examples, the antigen binding protein has a heavy chain variable region comprising SEQ ID NO: 7 or having an amino acid sequence that is at least 80% identical to the sequence or having 1 or 2 amino acids different from the sequence thereof, or fragments thereof.

In some examples, the antigen binding protein has a light chain variable region comprising SEQ ID NO: 8, or an amino acid sequence that is at least 80% identical to the sequence or having 1 or 2 amino acids different from the sequences thereof, or fragments thereof.

In some examples, the antigen binding protein has one or more CDR region encoded by a polynucleotide sequence selected from the group consisting of

(SEQ ID NO: 12)
GGCTTCTCAATCACCAGTGATTATGCC for CDRH1,
(SEQ ID NO: 13)
ATAAGTTACAGTGGTAGCACT for CDRH2,
(SEQ ID NO: 14)
GCAAGAGTTGTATCTACGTCTTTTGACTCC for CDRH3,
(SEQ ID NO: 15)
GAAAGTGTTGATAATTATGGCATTAGTTTT for CDRL1,
(SEQ ID NO: 16)
GTTGCATCC for CDRL2,
and
(SEQ ID NO: 17)
CAGCAAAGTAAGGAGGTTCCGTACACG for CDRL3,
or
polynucleotide that is at least 85%
identical to the sequence.

In some examples, the antigen binding protein has a heavy chain variable region encoded by a polynucleotide sequence comprising SEQ ID NO: 9, or a sequence that is at least 70% identical to the sequence.

In some examples, the antigen binding protein comprises a light chain variable region encoded by a polynucleotide sequence comprising SEQ ID NO: 10, or a sequence that is at least 70% identical to the sequence.

In some examples, the antigen binding protein is expressed on a chimeric antigen receptor T cell (CAR T cell) and/or a chimeric antigen receptor NK cell (CAR NK cell).

In another aspect, there is provided a polynucleotide encoding the antigen-binding protein as described herein.

In some examples, the polynucleotide comprises SEQ ID NO: 9 and/or 10.

In yet another aspect, there is provided a cloning or expression vector expressing the antigen binding protein of present disclosure or polynucleotide of of present disclosure.

In yet another aspect, there is provided a host cell comprising the vector as described herein.

In yet another aspect, there is provided a pharmaceutical composition comprising the antigen-binding protein as described herein and a suitable pharmaceutical excipient, diluent, carrier, or additive thereof.

In yet another aspect, there is provided antigen-binding protein or composition or pharmaceutical composition as described herein for use in therapy and/or immunotherapy and/or as a therapeutic agent.

In yet another aspect, there is provided antigen binding protein or pharmaceutical composition as described herein for use in treating a proliferative disease and/or TACI-positive tumour/cancer and/or multiple myeloma (MM) in a subject in need thereof.

In yet another aspect, there is provided the use of the antigen binding protein or composition or pharmaceutical composition as described herein in the manufacture of a medicament for treating a disease in a subject in need thereof, optionally the disease is a proliferative disease, optionally the disease is a TACI-positive tumour/cancer, and/or multiple myeloma (MM).

In yet another aspect, there is provided a method of treating a disease in a subject in need thereof, wherein the method comprises administering a therapeutically effective amount of the antigen binding protein or composition or pharmaceutical composition as described herein.

In yet another aspect, there is provided a method of neutralizing an autoimmune disease and/or a tumour and/or cancer cell in a subject in need thereof, wherein the method comprises administering a therapeutically effective amount of the antigen binding protein or composition or pharmaceutical composition as described herein into the subject.

### DEFINITIONS

The term “recombinant polypeptide” as used herein refers to a polypeptide that has been made using any recombinant DNA technique.

The term “fusion polypeptide” as used herein refers to a polypeptide comprising at least two domains that have been encoded by separate genes and joined such that the two domains are transcribed and translated as a single unit, thereby producing a single polypeptide.

The term “polypeptide” as used herein refers a single linear chain of any number of amino acid residues connected via peptide bonds.

The term “antibody” is used in the broadest sense and specifically covers monoclonal antibodies (including full-length monoclonal antibodies and smaller molecules derived therefrom) and polyclonal antibodies. The term “antibody” also encompasses monovalent and multi-valent antibodies, e.g., bivalent antibodies, trivalent antibodies, and tetravalent antibodies. The term “antibody” also encompasses monospecific and multispecific antibodies, e.g., bispecific antibodies, trispecific antibodies, tetraspecific antibodies, and the like. A multispecific antibody is an antibody capable of binding to two or more different antigens or two different epitopes of the same antigen.

As used herein, the term “sequence identity” refers to the percentage sequence identities that are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

At least 70% identical, or 75% identical, or 80% identical, or 85% identical, or 90% identical, or 95% identical, or 97% identical, or 98% identical, or 99% identical, or 100% identical to the sequence 95% identical” as employed herein is intended to refer to an amino acid sequence which over its full length is 70%, 75%, 80%, 85%, 90%, 95%, 97%, 98%, 99% identical or more to a reference sequence, such as 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical. Software programmes can be employed to calculate percentage identity.

The term “immunoglobulin fragment” as used herein refers to an antibody that may be, but are not limited to Fab, modified Fab, Fab′, modified Fab′, F(ab′)2, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example, Verma et al., 1998, Journal of Immunological Methods, 216, 165-181).

The term “neutralizing” with respect to an antigen-binding protein refers to the ability to bind to a target cell (such as a cancer cell) and recruit phagocytic cells to incur direct cell cytotoxicity towards the target cell or to excrete agents that may induce cell apoptosis.

The term “subject” as used herein includes patients and non-patients. The term “patient” refers to individuals suffering or are likely to suffer from a medical condition such as a proliferative disease such as a TACI-positive cancer or a multiple myeloma, while “non-patients” refer to individuals not suffering and are likely to not suffer from the medical condition. “Non-patients” include healthy individuals, non-diseased individuals and/or an individual free from a medical condition. The term “subject” includes humans and animals. Animals include murine and the like. “Murine” refers to any mammal from the family Muridae, such as mouse, rat, and the like.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as as a proliferative disease such as a TACI-positive cancer or a multiple myeloma), symptoms and disorders. A medical condition also includes a body's response to a disease or disorder, e.g., inflammation. Those in need of such treatment include those already with a medical condition as well as those prone to getting the medical condition or those in whom a medical condition is to be prevented.

The term “therapeutic agent’ as used herein refers to a drug, protein, peptide, gene, chemical compound or other pharmaceutically active ingredient.

A “vector” is any molecule or composition that has the ability to carry a nucleic acid sequence into a suitable host cell where e.g., synthesis of the encoded polypeptide can take place. Typically, and preferably, a vector is a nucleic acid that has been engineered, using recombinant DNA techniques that are known in the art, to incorporate a desired nucleic acid sequence (e.g., a nucleic acid of the invention). Expression vectors typically contain one or more of the following components (if they are not already provided by the nucleic acid molecules): a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the endpoints of a range are intended to illustrate a preferred range. Whenever a range has been described, it is intended that the range covers and teaches all possible sub-ranges as well as individual numerical values within that range. That is, the endpoints of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical endpoints. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of antibodies that specifically bind and/or detect the transmembrane activator and calcium-modulator and cyclophilin ligand (CAML) interactor (TACI)/TNFRSF13B are disclosed hereinafter.

The present disclosure provides an antigen-binding protein that specifically binds and/or detects the transmembrane activator and calcium-modulator and cyclophilin ligand (CAML) interactor (TACI)/TNFRSF13B.

In some examples, the antigen binding protein activates transcription factors such as, but is not limited to, AP-1, NF-κB, and the like.

In some examples, the antigen-binding protein is capable of modulating TACI-induced B cell activation and/or differentiation, optionally, the antigen-binding protein induces B cell activation and/or differentiation.

In some examples, the antigen-binding protein binds to and/or detects a portion/fragment of TACI/TNFRSF13B comprising a sequence as provided in NCBI accession no. NM_012452, or having an amino acid sequence that is at least 85% identical or 90% identical or 95% identical or 97% identical or 98% identical or 99% identical or 100% identical to the sequence:

(SEQ ID NO: 11)
MSGLGRSRRGGRSRVDQEERFPQGLWTGVAMRSCPEEQYWDPLLGTCMS
CKTICNHQSQRTCAAFCRSLSCRKEQGKFYDHLLRDCISCASICGQHPK
QCAYFCENKLRSPVNLPPELRRQRSGEVENNSDNSGRYQGLEHRGSEAS
PALPGLKLSADQVALVYSTLGLCLCAVLCCFLVAVACFLKKRGDPCSCQ
PRSRPRQSPAKSSQDHAMEAGSPVSTSPEPVETCSFCFPECRAPTQESA
VTPGTPDPTCAGRWGCHTRTTVLQPCPHIPDSGLGIVCVPAQEGGPGA.

In some examples, the antigen-binding protein has one or more CDR regions having an amino acid sequence that is at least 85% identical or 90% identical or 95% identical or 97% identical or 98% identical or 99% identical or 100% identical to the sequence selected from the group consisting of GFSITSDYA (SEQ ID NO: 1), ISYSGST (SEQ ID NO: 2), ARVVSTSFDS (SEQ ID NO: 3), ESVDNYGISF (SEQ ID NO: 4), VAS (SEQ ID NO: 5), and QQSKEVPYT (SEQ ID NO: 6), or sequences having 1 or 2 amino acid different from the sequences thereof.

Heavy chain
CDRH1:
(SEQ ID NO: 1)
GFSITSDYA
CDRH2:
(SEQ ID NO: 2)
ISYSGST
CDRH3:
(SEQ ID NO: 3)
ARVVSTSFDS
Light chain
CDRL1:
(SEQ ID NO: 4)
ESVDNYGISF
CDRL2:
(SEQ ID NO: 5)
VAS
CDRL3:
(SEQ ID NO: 6)
QQSKEVPYT

In some examples, the antigen binding protein comprises two or more CDR regions, or three or more CDR regions, or four or more CDR regions, or five or more CDR regions, or all CDR regions having sequences SEQ ID NO: 1, SEQ ID NO: 2, SEQ ID NO: 3, SEQ ID NO: 4, SEQ ID NO: 5, and SEQ ID NO: 6.

In one aspect, there is provided an antigen-binding protein that specifically binds and/or detects the transmembrane activator and calcium-modulator and cyclophilin ligand (CAML) interactor (TACI)/TNFRSF13B, wherein the antigen binding protein comprises a CDR sequence having GFSITSDYA (SEQ ID NO: 1) for CDRH1, ISYSGST (SEQ ID NO: 2) for CDRH2, ARVVSTSFDS (SEQ ID NO: 3) for CDRH3, ESVDNYGISF (SEQ ID NO: 4) for CDRL1, VAS (SEQ ID NO: 5) for CDRL2, and QQSKEVPYT (SEQ ID NO: 6) for CDRH1, or having amino acid sequences that are at least 85% identical to the sequences, or sequences having 1 or 2 amino acids different from the sequences thereof.

In some examples, the antigen-binding protein binds to one or more antigens or epitopes.

In some examples, the antigen-binding protein binds to two or more antigens or epitopes, or three or more antigens or epitopes, or four or more antigens or epitopes, or five or more antigens or epitopes. In some examples, the antigen-binding protein may be a multispecific antigen-binding protein. In some examples, the antigen-binding protein may be a bispecific or a tri-specific antigen-binding protein.

In some examples, the antigen binding protein is a bispecific antigen binding protein that binds and/or detects TACI and/or engages/activates other immune cells, such as NK cells, T lymphocytes, other B lymphocytes, and the like.

In some examples, the antigen binding protein may be expressed on a CAR-T cell or NK cell or T or NK cell-engaging bispecific/multispecific antibodies to treat proliferative diseases such as tumour or cancer, more specifically, TACI-positive cancers.

In some examples, the antigen binding protein is a bispecific antigen binding protein that binds and/or detects TACI and CD3.

In some examples, the antigen binding protein is a recombinant/fusion polypeptide comprising an immunoglobulin Fc fragment (region/domain), a protein capable of extending the half-life of the recombinant/fusion polypeptide (such as albumin, human albumin, and the like), linkers capable of enhancing binding valency (for example a rigid linker or a flexible linker), or combinations thereof, for example wherein the immunoglobulin Fc fragment is an IgG Fc fragment, such as a human IgG Fc fragment.

In some examples, the antigen binding protein is an antibody, optionally a monoclonal antibody.

In some examples, the antigen-binding protein is a humanized antibody.

In some examples, the antigen-binding protein is a neutralizing antibody.

In some examples, the antigen-binding protein as described herein may induce/mediate antibody-dependent cellular cytotoxicity (ADCC).

In some examples, the antigen binding protein as described herein can induce TACI-positive multiple myeloma cell (such as MMCL MM1R) death in the presence of white blood cells, such as PBMCs.

In some examples, the antigen binding protein has a heavy chain variable region having an amino acid sequence that is at least 80% identical, or 85% identical, or 90% identical, or 95% identical, or 97% identical, or 98% identical, or 99% identical, or 100% identical to the sequence comprising SEQ ID NO: 7 or having 1 or 2 amino acids different from the sequences thereof, or fragments thereof.

As disclosed herein SEQ ID NO: 7 is as follows

(SEQ ID NO: 7)
DVQLQESGPGLVKPSQSLSLTCTVSGFSITSDYAWHWIRQFPGNKLEWV
GYISYSGSTRYNPSLNSRISITRDTSKNQFFLQLKSVTTEDTATYFCAR
VVSTSFDSWGLGTTLTVSSAKTTPPSVYPLAPGSAAQTNSMVTLGCLVK
GYFPEPVTVTWNSGS, where underlines represent CDR
regions.

In some examples, the antigen binding protein has a heavy chain variable region comprising SEQ ID NO: 7 or having an amino acid sequence that is at least 80% identical to the sequence or having 1 or 2 amino acids different from the sequence thereof, or fragments thereof.

In some examples, the antigen binding protein has a light chain variable region having an amino acid sequence that is at least 80% identical, or 85% identical, or 90% identical, or 95% identical, or 97% identical, or 98% identical, or 99% identical, or 100% identical to the sequence comprising SEQ ID NO: 8 or having 1 or 2 amino acids different from the sequences thereof, or fragments thereof.

As disclosed herein SEQ ID NO: 8 is as follows

(SEQ ID NO: 8)
GIVLTQSPPSLAVSLGQRATISCRASESVDNYGISFMNWFQQKPGQPPK
LLIYVASNQGSGVPARFSGSGSGTDFSLNIHPMEEDDTAMYFCQQSKEV
PYTFGGGTKLEIKR, wherein underlines represent CDR
regions.

In some examples, the antigen binding protein has a light chain variable region comprising SEQ ID NO: 8, or an amino acid sequence that is at least 80% identical to the sequence or having 1 or 2 amino acids different from the sequences thereof, or fragments thereof.

In some examples, the antigen binding protein has one or more CDR region encoded by a polynucleotide sequence that is at least 85% identical or 90% identical or 95% identical or 97% identical or 98% identical or 99% identical or 100% identical to the sequence selected from the group consisting of

(SEQ ID NO: 12)
GGCTTCTCAATCACCAGTGATTATGCC,
(SEQ ID NO: 13)
ATAAGTTACAGTGGTAGCACT,
(SEQ ID NO: 14)
GCAAGAGTTGTATCTACGTCTTTTGACTCC,
(SEQ ID NO: 15)
GAAAGTGTTGATAATTATGGCATTAGTTTT,
(SEQ ID NO: 16)
GTTGCATCC,
and
(SEQ ID NO: 17)
CAGCAAAGTAAGGAGGTTCCGTACACG.
Heavy Chain
(CDRH1; SEQ ID NO: 12)
GGCTTCTCAATCACCAGTGATTATGCC
(CDRH2; SEQ ID NO: 13)
ATAAGTTACAGTGGTAGCACT.
(CDRH3; SEQ ID NO: 14)
GCAAGAGTTGTATCTACGTCTTTTGACTCC.
Light Chain
(CDRL1; SEQ ID NO: 15)
GAAAGTGTTGATAATTATGGCATTAGTTTT.
(CDRL2; SEQ ID NO: 16)
GTTGCATCC.
(CDRL3; SEQ ID NO: 17)
CAGCAAAGTAAGGAGGTTCCGTACACG.

In some examples, the antigen binding protein has one or more CDR region encoded by a polynucleotide sequence selected from the group consisting of

(SEQ ID NO: 12)
GGCTTCTCAATCACCAGTGATTATGCC for CDRH1,
(SEQ ID NO: 13)
ATAAGTTACAGTGGTAGCACT for CDRH2,
(SEQ ID NO: 14)
GCAAGAGTTGTATCTACGTCTTTTGACTCC for CDRH3,
(SEQ ID NO: 15)
GAAAGTGTTGATAATTATGGCATTAGTTTT for CDRL1,
(SEQ ID NO: 16)
GTTGCATCC for CDRL2,
and
(SEQ ID NO: 17)
CAGCAAAGTAAGGAGGTTCCGTACACG for CDRL3,
or
polynucleotide that is at least 85%
identical to the sequence.

In some examples, the antigen binding protein has a heavy chain variable region encoded by a polynucleotide sequence that is at least 70% identical, or 75% identical, or 80% identical, or 85% identical, or 90% identical, or 95% identical, or 97% identical, or 98% identical, or 99% identical, or 100% identical to the sequence comprising SEQ ID NO: 9.

As disclosed herein, SEQ ID NO: 9 is as follows:

(SEQ ID NO: 9)
GATGTGCAGCTTCAGGAGTCGGGACCTGGCCTGGTGAAACCTTCTCAGT
CTCTGTCCCTCACCTGCACTGTCTCTGGCTTCTCAATCACCAGTGATTA
TGCCTGGCACTGGATCCGGCAGTTTCCAGGAAACAAACTGGAGTGGGTG
GGCTACATAAGTTACAGTGGTAGCACTCGCTACAACCCATCTCTCAACA
GTCGAATCTCTATCACTCGAGACACATCCAAGAACCAGTTCTTCCTGCA
GTTGAAATCTGTGACTACTGAGGACACAGCCACATATTTTTGTGCAAGA
GTTGTATCTACGTCTTTTGACTCCTGGGGCCTAGGCACCACTCTCACAG
TTTCCTCAGCCAAAACGACACCCCCATCTGTCTATCCACTGGCCCCTGG
ATCTGCTGCCCAAACTAACTCCATGGTGACCCTGGGATGCCTGGTCAAG
GGCTATTTCCCTGAGCCAGTGACAGTGACCTGGAACTCTGGATCCCTGT
CCAGCGGTGTGCACACCTTCT, wherein underlined are CDR
regions.

In some examples, the antigen-binding protein has a heavy chain variable region encoded by a polynucleotide sequence comprising SEQ ID NO: 9, or a sequence that is at least 70% identical to the sequence.

In some examples, the antigen binding protein comprises a light chain variable region encoded by a polynucleotide sequence that is at least 70% identical, or 75% identical, or 80% identical, or 85% identical, or 90% identical, or 95% identical, or 97% identical, or 98% identical, or 99% identical, or 100% identical to the sequence comprising SEQ ID NO: 10.

(SEQ ID NO: 10)
GGCATTGTGCTGACCCAATCTCCACCTTCTTTGGCTGTGTCTCTAGGGC
AGAGGGCCACCATCTCCTGCAGAGCCAGCGAAAGTGTTGATAATTATGG
CATTAGTTTTATGAACTGGTTCCAACAGAAACCAGGACAGCCACCCAAA
CTCCTCATCTATGTTGCATCCAACCAAGGATCCGGGGTCCCTGCCAGGT
TTAGTGGCAGTGGGTCTGGGACAGACTTCAGCCTCAACATCCATCCTAT
GGAGGAGGATGATACTGCAATGTATTTCTGTCAGCAAAGTAAGGAGGTT
CCGTACACGTTCGGAGGGGGGACCAAGCTGGAAATCAAACGG,
wherein underlines represent CDR regions.

In some examples, the antigen binding protein comprises a light chain variable region encoded by a polynucleotide sequence comprising SEQ ID NO: 10, or a sequence that is at least 70% identical to the sequence.

In some examples, an antibody may be a bivalent Y-shaped molecule comprising two identical heavy chains and two identical light chains. Disulfide bonds link together the heavy and light chain pairs as well as the two heavy chains. Each chain consists of one variable domain that varies in sequence and is responsible for antigen binding, these are known as the VH and VL domains for the heavy and light chains, respectively. Each chain also consists of at least one constant domain. In the light chain, there is a single constant domain (CL) and in the heavy chain, there are at least three (CH1, CH2 and CH3), sometimes four (CH4) depending on the isotype. Five types of mammalian Ig heavy chains are known: α, δ, ε, γ, and μ, wherein the type of heavy chain defines the class (isotype) of the antibody. In humans, there are five different classes or isotypes of antibodies, including IgA (which includes IgA1 and IgA2), IgD, IgE, IgG (which includes subclasses IgG1, IgG2, IgG3 and IgG4) and IgM.

In some examples, the antibody comprises a heavy chain and a light chain variable regions fused together to form a single-chain variable domain antibody (scFv) or a single-chain variable domain with an Fc portion (i.e., an scFv-Fc, e.g., a minibody). In some examples, the antibody fragment is a divalent or bivalent single-chain variable fragment, engineered by linking two scFvs together either in tandem (i.e., tandem scFv), or such that they dimerize to form diabodies. In some examples, the antibody is a trivalent single chain variable fragment, engineered by linking three scFvs together, either in tandem or in a trimer formation, to form triabodies. In some examples, the antibody is a tetrabody single-chain variable fragment. In some examples, the antibody is a “linear antibody” which is an antibody comprising a pair of tandem Fd segments (VH-CH1-VH-CH1) that form a pair of antigen-binding regions.

In some examples, the immunoglobulin fragment may include but is not limited to a Fc region/domain, one or more CH regions/domains, a Fab, a Fab′, a F(ab′)2, a single chain Fv (ScFv) and/or Fv fragments, hinge regions/domain, as well as fragments or portions thereof.

In some examples, a recombinant/fusion polypeptide may also contain portions of immunoglobulin molecules or antibodies, such as including, but not limited to, all or portions of a constant heavy chain, a variable heavy chain, a constant light chain, a variable light chain, a hinge region, and/or an Fc domain of an Ig, as well as variants thereof. For example, a recombinant/fusion polypeptide as described herein may be combined with portions of a human IgG.

The amino acid composition of a light and/or heavy chain variable region of an antibody can be modified by amino acid insertion, deletion, or substitution. An amino acid “substitution” encompasses the replacement of an amino acid residue at a particular position in a polypeptide sequence with a different amino acid. In some examples, the substitution involves amino acid residue that is not naturally occurring at the particular position, either not naturally occurring within the organism or in any organism. Alternatively, one or more substitutions may be amino acid residues that are frequently occurring residues in sequences of similar related polypeptides in the same organism, e.g., conserved amino acid residues that occur frequently in sequences from other antibodies from the same species. Substitutions with conserved residues from naturally occurring sequences from the same species, e.g., human sequences, can reduce the chances of increased antigenicity for the polypeptides harboring one or more substitutions. In addition, conservation of charge (e.g., relatively higher frequency of the same positively charged, negatively charged, or uncharged species) compared to sequences from related polypeptides in the same organism is also considered in making the amino acid residue selections for substitution. These charge considerations can have an impact on maintaining the structure and/or stability of the antibody that comprises one or more amino acid substitutions. For purposes of modifying the pi of the antibody, the amino acid substitutions involve substituting an amino acid residue having a positive or negative charge with an amino acid residue having a neutral charge or vice versa. Exemplary substitutions include, but are not limited to: (1) substitution of a neutrally charged amino acid for a positively charged amino acid to increase the pi; (2) substitution of a negatively charged amino acid for a neutral or positively charged amino acid to increase the pi; (3) substitution of a neutrally charged amino acid for a negatively charged amino acid to decrease the pi; and (4) substitution of a positively charged amino acid with a neutral or negatively charged amino acid to decrease the pi.

Such substitutions, insertion, or deletions often involve gene manipulation or mutagenesis that modifies the original nucleic acid molecule encoding the polypeptide to be modified by inserting, deleting, or substituting at least one nucleotide, to produce a codon that encodes an amino acid residue of interest. More specifically, a codon encoding the original amino acid residue is replaced by a codon encoding the amino acid residue to be introduced by the modification. Such nucleic acid modifications can be carried out by those of skill in the art using well-known techniques such as site-directed mutagenesis or PCR mutagenesis.

In some examples, the amino acid substitutions, insertions, or deletions are introduced into one or more of the heavy chain polypeptides of the antibody of interest. In some examples, the amino acid substitutions, insertions, or deletions are introduced in the heavy chain variable region of the antibody of interest. The variable region of the heavy chain is subdivided into regions of hypervariability, termed complementarity-determining regions (CDR). The three CDRs in the variable regions of the heavy chain are designated CDR1, CDR2, and CDR3 for each of the variable regions. The CDRs are interspersed with more conserved regions termed framework regions (FR). These FR regions are specific to place in the proper spatial configuration the contact amino acid residues of the CDRs that are responsible for most of the binding capacity of the antibody. Each VH is composed of three CDRs and four FRs, arranged from amino-terminus to carboxy-terminus in the following order: FR1, CDR1, FR2, CDR2, FR3, CDR3, FR4. The exact boundaries of these CDRs have been defined differently according to different systems. The system described by Kabat (Kabat et al., Sequences of Proteins of Immunological Interest (National Institutes of Health, Bethesda, Md. (1987) and (1991), which is hereby incorporated by reference in its entirety), which is used herein when referencing residues of the heavy chain variable region, not only provides an unambiguous residue numbering system applicable to any variable region of an antibody but also provides precise residue boundaries defining the three CDRs. These CDRs are referred to as Kabat CDRs.

In some examples, the antigen-binding protein is an antibody having an isotype selected from the group consisting of IgG, IgA, IgM, IgE, IgD, IgY, and the like.

In some examples, the antigen-binding protein is an antibody selected from the group consisting of IgG1, IgG2, IgG3, IgG4, IgA1, IgA2, and the like.

In some examples, the antigen binding protein is expressed on a chimeric antigen receptor T cell (CAR T cell) and/or a chimeric antigen receptor NK cell (CAR NK cell).

In some aspects, there is provided a method of detecting TACI-positive cells (such as TACI positive tumour or cancer) comprising contacting a cell with the antigen-binding protein as disclosed herein. In some examples, the method of detecting TACI-positive cells may be a method of diagnosing a disease.

In another aspect, there is provided a polynucleotide encoding the antigen-binding protein as described herein.

In some examples, the polynucleotide comprises SEQ ID NO: 9 and/or 10.

In yet another aspect, there is provided a vector expressing the antigen-binding protein or polynucleotide as described herein. In some examples, there is provided a cloning or expression vector expressing the antigen binding protein or polynucleotide as disclosed herein.

In some examples, the vector is a tricistronic vector expressing the constant region of an IgG, for example, the constant region of trastuzumab IgG1.

In some examples, the vector further comprises an antibiotic-resistant gene (such as a zeocin-resistant gene) to produce a chimeric antigen-binding protein.

In yet another aspect, there is provided a host cell comprising the vector as described herein.

In yet another aspect, there is provided a composition comprising the antigen-binding protein as described herein.

In yet another aspect, there is provided a pharmaceutical composition comprising the antigen-binding protein as described herein and a suitable pharmaceutical excipient/carrier/additive thereof. In some examples, there is provided a pharmaceutical composition comprising the antigen-binding protein as disclosed herein and a suitable pharmaceutical excipient, diluent, carrier, or additive thereof.

In yet another aspect, there is provided the antigen binding protein or composition or pharmaceutical composition as described herein for use in therapy and/or immunotherapy and/or as a therapeutic agent.

In some examples, the antigen binding protein or composition or pharmaceutical composition as described herein is for use in treating a proliferative disease and/or TACI-positive tumour/cancer and/or multiple myeloma (MM) in a subject in need thereof.

In yet another aspect, there is provided the use of the antigen binding protein or composition or pharmaceutical composition of as described herein in the manufacture of a medicament for treating a disease in a subject in need thereof.

In another aspect, there is provided a method of treating a disease in a subject in need thereof, wherein the method comprises administering a therapeutically effective amount of the antigen binding protein or composition or pharmaceutical composition of the present disclosure.

In some examples, the disease is a proliferative disease.

In some examples, the proliferative disease is a plasma cell-related disease such as an autoimmune disease and/or a tumour and/or cancer.

In some examples, the disease is a TACI-positive tumour/cancer and/or multiple myeloma (MM).

In yet another aspect, there is provided a method of neutralizing an autoimmune disease and/or a tumour and/or cancer cell in a subject in need thereof, wherein the method comprises administering a therapeutically effective amount of the antigen binding protein or composition or pharmaceutical composition of the present disclosure.

In some examples, the disease is a proliferative disease.

In some examples, the proliferative disease is a plasma cell-related disease such as an autoimmune disease and/or a tumour and/or cancer.

In some examples, the disease is a TACI-positive tumour/cancer, and/or multiple myeloma (MM).

In some examples, the antigen binding protein or composition is administered to the subject by mode of administrations known in the art, including but not limited to intramuscularly, subcutaneously, intravenously, intraarterially, intraarticularly, intraperitoneally, intranasally, parenterally, and the like.

In some examples, the subject is a mammal, such as a monkey, rabbit, mouse, rat, pig, or dog. In some examples, the subject is a human.

In some examples, the antigen-binding protein may be provided as a kit. Therefore, there is provided kits comprising the antigen-binding protein as disclosed herein. In some examples, the kits may further comprise instructions to use the antigen-binding proteins as disclosed herein in the methods thereof.

Also disclosed are antigen-binding protein/antibody/method/use/composition as described herein.

### BRIEF DESCRIPTION OF FIGURES

Example embodiments of the disclosure will be better understood and readily apparent to one of ordinary skill in the art from the following discussions and, if applicable, in conjunction with the figures. It should be appreciated that other modifications to structural regions, constant regions, or variable regions of the antigen-binding protein may be made without deviating from the scope of the invention. Example embodiments are not necessarily mutually exclusive as some may be combined with one or more embodiments to form new exemplary embodiments. The example embodiments should not be construed as limiting the scope of the disclosure.

FIG. 1 shows the generation of anti-human TACI monoclonal antibodies using DNA vaccination and hybridoma technologies. (A) shows FACS analysis of TACI-binding of three clones of anti-TACI antibodies (T11C5, T20F2, and T21D11) using TACI-expressing HEK 293 T (HEK 293-TACI) cells (blue line). Parental HEK 293T cells (red line) were used as the negative control. (B) FACS analysis of TACI-binding of antibody clone T11C5 using TACI-expressing MMCLs-MM1R and RPMI 8226. TACI-negative MMCLs-KMS28 and H929—were used as controls.

FIG. 2 shows Ig heavy and light chain sequences and specificity validation of anti-TACI monoclonal antibody T11C5 (A) DNA sequences of Ig heavy and light chains of mouse anti-human TACI antibody T11C5. (B) Protein sequences of Ig heavy and light chains of T11C5. (C) FACS analysis of TACI-binding of chimeric antibody T11C5, which comprises the variable regions of mouse T11C5 antibody and the constant regions of human IgG1 antibodies, using TACI-expressing HEK 293T cells. BCMA-expressing HEK 293T cells were used as the negative control. (D) FACS analysis of the killing of TACI-expressing MMCL MM1R human PBMCs mediated by a T cell-redirecting and TACI-targeting bispecific antibody (BsAb) harboring the Ig variable sequences of T11C5.

### EXPERIMENTAL SECTION

### Generation of Mouse Anti-Human TACI Antibodies

Balb/c mice (InVivos Pte Ltd, Singapore) were immunized with DNA plasmid in lactated Ringer's solution via hydrodynamic tail vein (HTV) injection. The mice were first primed with an injection of 10 μg mFlt3L plasmid DNA. Three days post-priming, the mice were immunized weekly with 50 μg hTACI plus 2.5 μg mGM-CSF and 2.5 μg mIL-21 DNA plasmid via HTV injection for 4 weeks. After resting for 2 months, the mice were boosted with the last dose of HTV immunization 3 days before they were sacrificed.

Splenocytes were harvested from the immunized mice and fused with SP2/0 mouse myeloma cells (ATCC, USA) using a ClonaCell™-HY Hybridoma Kit (Stem Cell Technologies, Canada). Anti-hTACI antibody-producing clones were screened by flow cytometry using hTACI-expressing 293 T cells.

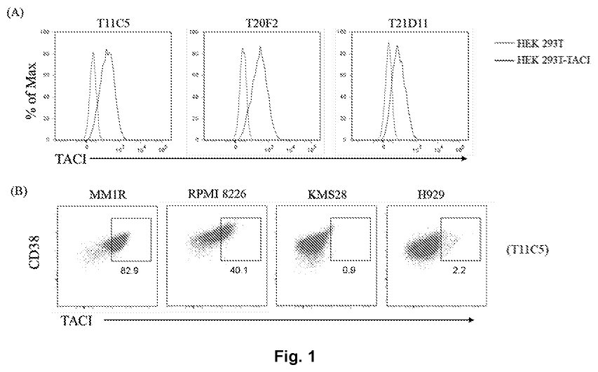

Totally, three positive clones of antibodies were obtained, namely T11C5, T20F2, and T21D11, specifically binding the TACI-expressing HEK 293T (HEK 293T-TACI) cells (FIG. 1A). Next, several multiple myeloma cell lines (MMCLs), such as MM1R, RPMI 8226, KMS28, and H929, were stained. The inventors found that the antibodies as disclosed herein, such as T11C5, could specifically stain TACI-expressing MM1R and RPMI 8226 but not TACI-negative KMS28 and H929 (FIG. 1B).

### Anti-Human TACI Antibody T11C5 Sequencing

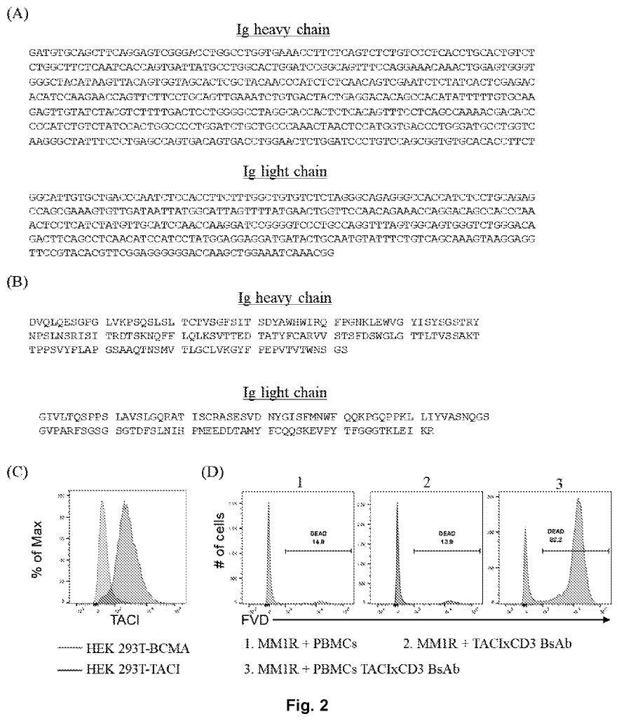

After subcloning the positive clones of hybridoma cells, genes encoding the variable (V) region of the immunoglobulin (Ig) heavy and light chains (HC and LC) were amplified by PCR and cloned using TArget Clone-Plus kit (TOYOBO) followed by sequencing. The cDNA and the translated protein sequences of VH and VL fragments of clone T11C5 are shown in FIGS. 2A and 2B, respectively.

### Specificity of Anti-Human TACI Antibody T11C5

The inventors further cloned the VH and VL gene fragments into a tricistronic vector expressing the constant regions of trastuzumab IgG1 and a zeocin-resistant gene to produce the chimeric anti-hTACI monoclonal antibody. FACS analysis confirmed that the chimeric anti-TACI antibody could specifically bind TACI (FIG. 2C). In addition, the inventors generated a bispecific antibody (bsAb) TACIxCD3 and found that the bsAb could kill TACI-positive MMCL MM1R in the presence of PBMCs (FIG. 2D).

1. An antigen-binding protein that specifically binds and/or detects the transmembrane activator and calcium-modulator and cyclophilin ligand (CAML) interactor (TACI)/TNFRSF13B, wherein the antigen binding protein comprises a CDR sequence having GFSITSDYA (SEQ ID NO: 1) for CDRH1, ISYSGST (SEQ ID NO: 2) for CDRH2, ARVVSTSFDS (SEQ ID NO: 3) for CDRH3, ESVDNYGISF (SEQ ID NO: 4) for CDRL1, VAS (SEQ ID NO: 5) for CDRL2, and QQSKEVPYT (SEQ ID NO: 6) for CDRH1, or having amino acid sequences that are at least 85% identical to the sequences, or sequences having 1 or 2 amino acids different from the sequences thereof.

2. The antigen binding protein of claim 1, wherein the antigen binding protein activates transcription factors AP-1 and/or NF-κB.

3. The antigen binding protein of claim 1, wherein the antigen binding protein is capable of modulating TACI-induced B cell activation and/or differentiation.

4. The antigen binding protein of claim 1, wherein the antigen binding protein is a multispecific antigen binding protein that binds and/or detects TACI and/or engages/activates other immune cells, optionally wherein the antigen binding protein is a bispecific antigen binding protein that binds and/or detects TACI and CD3.

5. The antigen binding protein of claim 1, wherein the antigen binding protein is a monoclonal antibody.

6. The antigen binding protein of claim 1, wherein the antigen binding protein is a neutralizing antibody and/or a humanized antibody.

7. The antigen binding protein of claim 1, wherein the antigen binding protein has a heavy chain variable region comprising SEQ ID NO: 7 or having an amino acid sequence that is at least 80% identical to the sequence or having 1 or 2 amino acids different from the sequence thereof, or fragments thereof.

8. The antigen binding protein of claim 1, wherein the antigen binding protein has a light chain variable region comprising SEQ ID NO: 8, or an amino acid sequence that is at least 80% identical to the sequence or having 1 or 2amino acids different from the sequences thereof, or fragments thereof.

9. The antigen binding protein of claim 1, wherein the antigen binding protein has one or more CDR region encoded by a polynucleotide sequence selected from the group consisting of
(SEQ ID NO: 12)
GGCTTCTCAATCACCAGTGATTATGCC for CDRH1,
(SEQ ID NO: 13)
ATAAGTTACAGTGGTAGCACT for CDRH2,
(SEQ ID NO: 14)
GCAAGAGTTGTATCTACGTCTTTTGACTCC for CDRH3,
(SEQ ID NO: 15)
GAAAGTGTTGATAATTATGGCATTAGTTTT for CDRL1,
(SEQ ID NO: 16)
GTTGCATCC for CDRL2,
and
(SEQ ID NO: 17)
CAGCAAAGTAAGGAGGTTCCGTACACG for CDRL3,
or
polynucleotide that is at least 85%
identical to the sequence.

10. The antigen binding protein of claim 1, wherein the antigen binding protein has a heavy chain variable region encoded by a polynucleotide sequence comprising SEQ ID NO: 9, or a sequence that is at least 70% identical to the sequence.

11. The antigen binding protein of claim 1, wherein the antigen binding protein comprises a light chain variable region encoded by a polynucleotide sequence comprising SEQ ID NO: 10, or a sequence that is at least 70% identical to the sequence.

12. The antigen binding protein of claim 1, wherein the antigen binding protein is expressed on a chimeric antigen receptor T cell (CAR T cell) and/or a chimeric antigen receptor NK cell (CAR NK cell).

13.-16. (canceled)

17. A pharmaceutical composition comprising
the antigen-binding protein of claim 1, or
a polynucleotide encoding the antigen binding protein of claim 1, and a suitable pharmaceutical excipient, diluent, carrier, or additive thereof.

18.-20. (canceled)

21. A method of treating a disease in a subject in need thereof, wherein the method comprises administering a therapeutically effective amount of the antigen binding protein or composition or pharmaceutical composition of claim 1 into the subject.

22. (canceled)

23. The method of claim 21, wherein the disease is an autoimmune disease and/or a tumour and/or cancer.

24. The pharmaceutical composition of claim 17, wherein the polynucleotide comprises SEQ ID NO: 9 and/or 10.

25. The pharmaceutical composition of claim 17, further comprising a cloning or expression vector.

26. The pharmaceutical composition of claim 17, further comprising a host cell.


SEQUENCE LISTING (17 sequences)

SEQ ID NO: 1 - protein, 9 aa, synthetic construct
GFSITSDYA

SEQ ID NO: 2 - protein, 7 aa, synthetic construct
ISYSGST

SEQ ID NO: 3 - protein, 10 aa, synthetic construct
ARVVSTSFDS

SEQ ID NO: 4 - protein, 10 aa, synthetic construct
ESVDNYGISF

SEQ ID NO: 5 - skipped

SEQ ID NO: 6 - protein, 9 aa, synthetic construct
QQSKEVPYT

SEQ ID NO: 7 - protein, 162 aa, synthetic construct
DVQLQESGPGLVKPSQSLSLTCTVSGFSITSDYAWHWIRQFPGNKLEWVGYISYSGSTRYNPSLNSRISITRDTSKNQFFLQLKSVTTEDTATYFCARVVSTSFDSWGLGTTLTVSSAKTTPPSVYPLAPGSAAQTNSMVTLGCLVKGYFPEPVTVTWNSGS

SEQ ID NO: 8 - protein, 112 aa, synthetic construct
GIVLTQSPPSLAVSLGQRATISCRASESVDNYGISFMNWFQQKPGQPPKLLIYVASNQGSGVPARFSGSGSGTDFSLNIHPMEEDDTAMYFCQQSKEVPYTFGGGTKLEIKR

SEQ ID NO: 9 - DNA, 511 bp, synthetic construct
  source 1..511: mol_type = other DNA
gatgtgcagcttcaggagtcgggacctggcctggtgaaaccttctcagtctctgtccctcacctgcactgtctctggcttctcaatcaccagtgattatgcctggcactggatccggcagtttccaggaaacaaactggagtgggtgggctacataagttacagtggtagcactcgctacaacccatctctcaacagtcgaatctctatcactcgagacacatccaagaaccagttcttcctgcagttgaaatctgtgactactgaggacacagccacatatttttgtgcaagagttgtatctacgtcttttgactcctggggcctaggcaccactctcacagtttcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttct

SEQ ID NO: 10 - DNA, 336 bp, synthetic construct
  source 1..336: mol_type = other DNA
ggcattgtgctgacccaatctccaccttctttggctgtgtctctagggcagagggccaccatctcctgcagagccagcgaaagtgttgataattatggcattagttttatgaactggttccaacagaaaccaggacagccacccaaactcctcatctatgttgcatccaaccaaggatccggggtccctgccaggtttagtggcagtgggtctgggacagacttcagcctcaacatccatcctatggaggaggatgatactgcaatgtatttctgtcagcaaagtaaggaggttccgtacacgttcggaggggggaccaagctggaaatcaaacgg

SEQ ID NO: 11 - protein, 293 aa, synthetic construct
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 12 - DNA, 27 bp, synthetic construct
  source 1..27: mol_type = other DNA
ggcttctcaatcaccagtgattatgcc

SEQ ID NO: 13 - DNA, 21 bp, synthetic construct
  source 1..21: mol_type = other DNA
ataagttacagtggtagcact

SEQ ID NO: 14 - DNA, 30 bp, synthetic construct
  source 1..30: mol_type = other DNA
gcaagagttgtatctacgtcttttgactcc

SEQ ID NO: 15 - DNA, 30 bp, synthetic construct
  source 1..30: mol_type = other DNA
gaaagtgttgataattatggcattagtttt

SEQ ID NO: 16 - skipped

SEQ ID NO: 17 - DNA, 27 bp, synthetic construct
  source 1..27: mol_type = other DNA
cagcaaagtaaggaggttccgtacacg